(19) United States
(12) Patent Application Publication
(10) Pub. No.: US 2007/0005146 A1
(43) Pub. Date: Jan. 4, 2007

(54) Hip stem for receiving intramedullary nail

(75) Inventors: Ide Christiaan Heyligers, Maastricht (NL); Bernd Peter Ludwig Grimm, Trier (DE); Gearoid Walsh, Co. Clare (IE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/172,290

(22) Filed: Jun. 30, 2005

(51) Int. Cl. A61F 2/30; A61F 2/36

(52) U.S. Cl. 623/23.46; 623/22.41; 623/22.46

(57) ABSTRACT

A method of operating on a joint having a medullary canal, including the steps of removing a first rod that is disposed within a bore of a joint prosthesis. The first rod having a distal tip that extends past the joint prosthesis when the joint prosthesis is implanted in the medullary canal of a bone. Next, a second rod is placed within the bore of the joint prosthesis. The second rod has a second distal tip that extends distally further into the medullary canal of the bone as compared to the distal tip of the first rod when the first rod is positioned within the joint prosthesis.

### BACKGROUND OF THE INVENTION

The present invention relates generally to systems, kits and methods for joint replacement using multiple components. In one embodiment, the present invention includes as components a head, a neck and a stem.

Artificial joint prostheses are widely used today, restoring joint mobility to patients affected by a variety of conditions, including congenital, degenerative, iatrogenic and traumatic afflictions of the joints. The satisfactory performance of these devices can be affected not only by the design of the component itself, but also by the surgical positioning of the implanted component and the long-term fixation of the device. Improper placement or positioning of the device can adversely affect the goal of satisfactorily restoring the clinical bio-mechanics of the joint as well as impairing adequate fixation of the component when implanted within the medullary joint prosthesis is to restore the extremity distal to the diseased and/or damaged joint to normal function.

As one example, an implantable joint prosthesis can be used to provide an artificial hip. When the prosthesis is situated in this position, significant forces such as axial, bending, and rotational forces are imparted to the device. The prosthesis must endure these forces while remaining adequately fixed within the medullary canal, because adequate fixation is necessary to ensure the implant's proper functioning and a long useful life. Early designs of artificial hip components relied primarily on cemented fixation. These cements, such as pqlymethylmethacrylate, were used to anchor the component within the medullary canal by acting as a grouting agent between the component and the endosteal (inner) surface of the bone. While this method of fixation by cement provides immediate fixation and resistance to the forces encountered, and allows the surgeon to effectively position the device before the cement sets, it is not without problems. Over time, the mechanical properties and the adhesive properties of the bone cement degrade; eventually the forces overcome the cement and cause the components to become loose due to a failure at the cement/bone or cement/stem interface. Alternative approaches to address the issue of cement failure include both biological ingrowth and press-fit stems.

Stems designed for biological ingrowth typically rely on the bone itself to grow into a specially prepared surface of the component, resulting in firmly anchoring the device within the medullary canal. A shortfall of this approach is that, in contrast to components that utilize cement fixation, surfaces designed for biological ingrowth do not provide for immediate fixation because it takes time for the bone to grow into the specially prepared surface. Press-fit stems precisely engineered to fit within a surgically prepared medullary canal may or may not have specially prepared surfaces and typically rely on an interference fit of some degree of the component within the medullary canal of the bone to achieve stable fixation.

In either case, whether cemented fixation implants, press-fit implant or biological ingrowth implants are used the need often arises to replace at least a portion of the implant. Prior art designs often require the entire implant be replaced even if only a portion of the implant fails. Similarly, the entire implant may have to be replaced even if the implant is fine but certain conditions surrounding the implant have changed. This is often due to the implant suffering from a decrease in distal support cause for a myriad of reasons. In such an instance, with prior art technology a surgeon is forced to operate and conduct an entire second operation.

The surgeon must first remove the entire first implant and replace it with a second implant. Unfortunately, the elderly, who are most likely to suffer from such a complication, are in the class of people who are most susceptible to harm caused by extended and plural surgeries.

Therefore there is a need to provide a more efficient method of replacing a joint prosthesis or portion thereof while minimizing the time spent on the operating table for the patient as will as reducing the recovery time post surgery.

For ease of reference, the present application refers to the distal position of an element as being the part of the element further away from the heart of an animal. And the proximal position of an element is closer to the heart as compared to the reference point that the part of the element is measured against.

### SUMMARY OF THE INVENTION

In one embodiment of the present invention a joint prosthesis kit having a plurality of stems is provided. Each of the stems may have a first side, a second side, a proximal end, a distal end, and a bore extending from the proximal end to the distal end. The bore is defined by an interior wall. The stem is adapted configured so as to be insertable within a medullary canal of a bone.

The joint prosthesis kit may further include a first rod having a distal tip capable of being inserted into the bore of the proximal end of the stem and housed in the bore. The distal tip of the first rod extends past the distal end of the stems such that the first rod extends farther into the medullary canal of the bone than the stems. The tip further includes a second rod having a distal tip capable of being inserted into the bore in the proximal end of the stem. The first rod being replaceable by the second rod such that the distal tip of the second rod extends distally further into the medullary canal of the bone when the second rod is disposed within the stem and the medullary canal as compared to the distal tip of the first rod when the first rod is disposed within the stem and the medullary canal.

The joint prosthesis kit may further include a coupling element for coupling the first and second rods to the proximal end of the stems.

In one preferred embodiment, the stem is implanted into the medullary canal of a femur.

The joint prosthesis kit may also include a neck and a head. The neck being disposed at the proximal end of the stem and the head extending outwardly from the neck and away from the distal end of the stem. The head may be removably attached to the neck.

The first rod may also include a proximal end with a cap position near the proximal of the cap. Such that when the first rod is placed within the bore of the stem, the cap abuts the proximal end of the stem to limit a depth to which the first rod may extend into the medullary canal of the bone.

The interior wall of the stem may include a cylindrical portion, a tapered portion, a noncylindrical portion or various other shapes such as triangular, planar, and so forth. The stems of the kit may include a second interior wall defining a second bore capable of receiving an additional rod. The additional rod may be constructed so as to be replaceable by a fourth rod similar to the placeability of the first rod by the second rod.

The present invention also contemplates various apertures extending from one side of the rod to the other side of the rod. The apertures are able to receive fixation elements to thereby secure the bone to the stem by placing the fasteners through the bone into the aperture of the stem.

The present invention also discloses a method of operating on a joint having a medullary canal, including the steps of removing a first rod disposed within a bore of a joint prosthesis. The first rod having a distal tip that extends past the joint prosthesis when the joint prosthesis is implanted into the medullary canal of the bone. A second rod may then be placed in the bore of the joint prosthesis. The second rod having a second distal tip that extends distally further into the medullary canal of the bone as compared to the distal tip of the first rod when the first rod is positioned within the joint prosthesis in the medullary canal of the bone. The joint prosthesis may be a hip implant. The step of placing the second rod in the bore of the joint prosthesis may include introducing the second rod into the bore of the joint prosthesis at the proximal end of the joint prosthesis and sliding the second rod from the proximal end of the stem in a direction toward the distal end of the joint prosthesis.

### BRIEF DESCRIPTION OF THE DRAWINGS

FIG. 1 is a cross-sectional side view of an embodiment of a stem used in conjunction with the present invention;

FIG. 2 is a cross-sectional side view of an embodiment of a rod used in conjunction with the present invention;

FIG. 3 is a cross-sectional side view of an embodiment of a rod being placed within an embodiment of a stem according to the present invention;

FIGS. 4a and 4b are side views of an embodiment of the present invention;

FIG. 5a is an illustration of a bone for which the present invention is adapted to be insertable into;

FIG. 5b is an illustration of the bone of FIG. 5a receiving an embodiment of the present invention therewithin;

FIG. 6 is a cross-sectional side view of an embodiment of the present invention housed within a bone;

FIG. 7 is a cross-sectional side view of an altered embodiment of the present invention housed within a bone;

FIGS. 8-14 are illustrations of alternate embodiments of the present invention;

FIGS. 15-18 are illustrations of alignment devices used in conjunction with the invention; and

FIGS. 19a-19f are illustrations of coupling elements used in conjunction with the present invention.

### DETAILED DESCRIPTION

The present invention is suitable for a joint implant which allows access to a canal of a hollow bone by having a bore hole or recess included in the implant. The bore hole may be hollow or have any other form of a cavity or recess or a combination thereof. In a preferred embodiment, the access can be used to enter exchangeable modular elements to either just occlude the cavity, or for implant stabilization, for example, in order to increase distal support or tailor stiffness. The exchangeable modular elements may also be used for the treatment of periprosthetic fractures either during, intra or postoperatively as the implant remains in place. Although the present invention is suitable for many joint implants where access to a medullary canal is beneficial, the present invention is particularly advantageous for use in conjunction with an artificial hip implant and as such this description will reference a hip prosthesis for illustrated purposes.

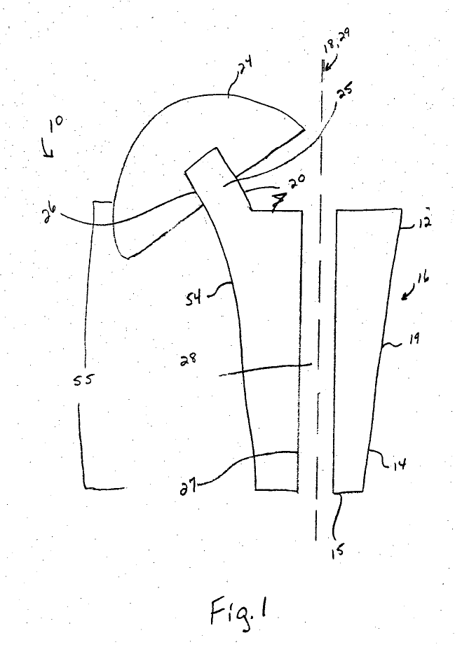

Referring to FIG. 1, there is shown a first embodiment of the present invention, which preferably includes a femoral stem 10 having a proximal end 12 and a distal end 14. A distal tip 15 is located at the far side of distal end 14. The femoral stem 10 also preferably includes an tapered portion 16, extending between proximal end 12 and distal tip 15, having a longitudinal axis 18. The tapered portion 16 is designed for insertion into a surgically prepared medullary cavity of a long bone, as will be described below. The femoral stem 10 further includes a neck portion 20 which may be integrally connected to tapered portion 16, as shown in FIG. 1. In alternate embodiments, neck portion 20 and tapered portion 16 may be modularly connected thereby permitting a plurality of neck portions to be interchanged with one or more tapered portions 16. In the instance where the two portions are independent from one another, the two portions may be assembled and joined together by any locking means know to those in the art.

The prosthesis may also include a modular head 24 attached to the neck 20 of either the one-piece integral tapered portion 16 or to the non-integral neck, typically by a Morse taper. For example, as shown in FIG. 1, neck 20 may include a spigot 25 extending upwardly away from tapered portion 16 and head 24 may include a tapered cavity 26. The preferably tapered spigot 25 may be received within tapered cavity 26 and locked thereto either by a Morse taper or using any method known in the art to lock head 24 to neck 20. The assembly of the head/neck and femoral stem 10 can occur prior to packaging, immediately prior to implantation or during implantation.

Additional aspects of the tapered portion 16 may include a midshaft 19, as shown in FIG. 1. Midshaft 19 extends between proximal end 12 and distal end 14, thereby providing the required shape for filling transition between the two elements. For instance, midshaft 19 and distal end 14 of the tapered portion 16 are preferably generally either cylindrical or conical in shape or a combination of both.

As shown in FIG. 1, femoral stem 10 includes a bore 28 preferably extending from the proximal end 12 of the femoral stem 10 to the distal end 14 of the femoral stem, thereby defining a continuous pathway through the femoral stem. Bore 28 is at least partially defined by interior wall 27. The purpose of the bore 28 will become apparent below. Bore 28 has a longitudinal axis 29, which as shown in FIG. 1, is preferably co-axial with axis 18 or may be substantially parallel to the longitudinal axis 18 of femoral stem 10. The bore 28 permits access to the medullary canal of a long bone, i.e., preferably the femur as discussed with reference to FIG. 1, when the femoral stem 10 is implanted.

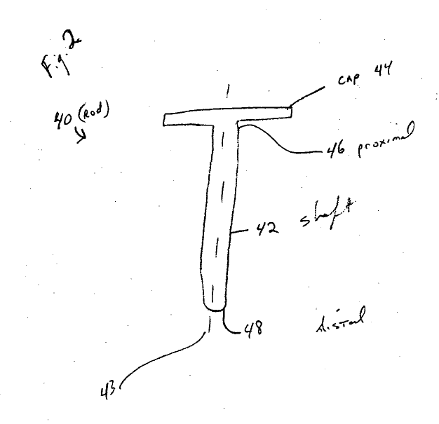

A rod 40, for use in conjunction with the femoral stem 10, is shown in FIG. 2. Rod 40 preferably includes a cylindrical shaft 42 having a central a longitudinal axis 43. Rod 40 preferably includes a cap 44 positioned near a proximal end 46 of the rod. The rod 40 also includes a distal end or tip 48 remote from proximal end 46. In the preferred embodiment end 48 is inwardly tapered at the tip.

In a method of assembly, rod 40 either after the tapered portion 16 has been implanted within the medullary canal of the femur or in conjunction with implanting the femoral stem 10, may be received within bore 28 of the femoral stem 10. In a preferred embodiment of the present invention, the distal end 48 of rod 40 may extend distally further into the medullary canal of the femur than the distal end of the femoral stem 10, when the two elements are implanted. By extending distally further, rod 40 provides increased distal support to the implant while also minimizing or at least reducing the amount of bone and material that must be removed in order to implant the femoral stem 10. This is due to the rod 40 being smaller in size than the femoral stem in a direction perpendicular to the longitudinal axes 18, 43. In an alternate embodiment, rod 40 may not extend distally further than the distal end of the femoral stem 10.

Rod 40 may function to occlude the bore against debris migration, which only requires the rod be snuggly captured within bore 28 as opposed to extending beyond the stem. And rod 40 can stiffen the prosthesis by adding stability to it. Also, rod 40 can be designed to guide the prosthesis, i.e. femoral stem 10 into proper position during insertion. This is particularly useful in the situation where the femoral stem is hammered into the intermedullary canal of the femur. The rod 40 further may prevent varus/valgus tilt should the femoral stem 10 move or migrate postoperatively.

In one embodiment of the present invention, as shown in FIGS. 1-3, bore 28 of femoral stem 10 and shaft 42 of rod 40 are substantially cylindrical. Further, shaft 42 preferably has a radius that is at least slightly less than the radius of bore 28, defined by interior wall 27. The rod 40 may be slidably received within the bore 28 or may include external threads (not shown) which engage internal threads (not shown) of interior wall 27. Various other communication and engaging mechanisms may be utilized without deviating from the scope of the invention.

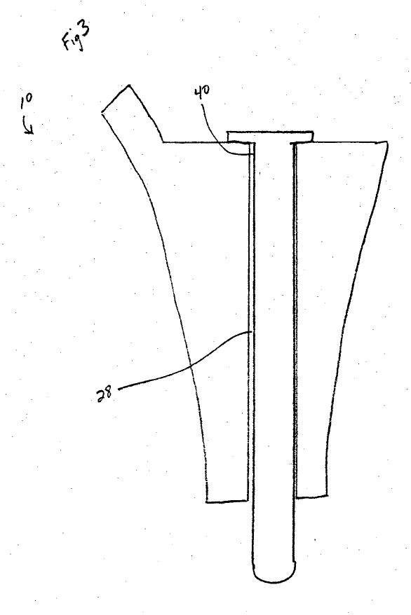

As shown in FIGS. 4a and 4b, midshaft 19 may include longitudinal ridges 50 with integrating flutes 51 forming sidewalls 53 to aid in positioning of the femoral stem 10 in a femur and to provide increased rotational stability of the femoral stem in the bone.

The flutes 51 may be formed by removing a minimal amount of material from the outer surface of the femoral stem 10. Ridges 50 may be separated from flutes 51 by raised walls 53. In the embodiment shown in FIGS. 4a and 4b, raised walls 53 are substantially orthogonal to the ends of flutes 51 and ridges 50. In an alternate embodiment, ridges 50 may be blended into the larger diameter of the femoral stem 10 while the flutes 51 blend into a smaller diameter of femoral stem 10 thereby resulting in a smooth transition of the stemmed surface similar to a sinusoidal curve.

In one preferred embodiment, ridges 51 may be parallel to one another and to the longitudinal axis 18 of femoral stem 10 to allow for stem insertion. The bone surface and resulting fixation can also be controlled by the depth of the flutes 51 defined by a height of the raised walls 53.

The location of midshaft 19 on the femoral stem 10 may be varied. Specifically, tapered portion 16 may be varied on different stem components to permit the surgeon greater flexibility in selecting the proper component for the patient's anatomy.

The midshaft 19 preferably blends into the relatively larger proximal end 12 of femoral stem 10. The proximal end 12 is larger so as to fill the larger proximal end of the bone canal. Thus, midshaft 19 tends to increase in size and change shape so as to correspond with the bone contour of the canal being larger. For this reason, a medial side 54 of the femoral stem widens as it approaches the proximal end 12 of the femoral stem 10.

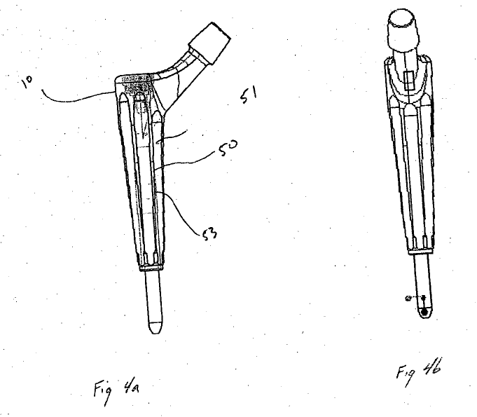

In a surgical method for using the joint replacement system of the present invention the proximal joint head of the femur is resected. The proximal joint head maybe resected as shown in U.S. Pat. No. 5,607,431, the disclosure of which is hereby incorporated by reference herein. A femur 60 with a resected proximal joint head is shown in FIG. 5a. The cortical bone of the femur is denoted as 62 and the cancellous bone portion is denoted as 63.

As shown in FIG. 5b, after the osteotomy, a reamer 64 is placed into the medullary canal 65 of the femur 60 and moved distally to remove the required amount of bone needed for implantation of the femoral stem 10. In addition, portions of the bone may be chiseled away if required. After reamer 64 is inserted in the medullary cavity and driven distally to ream out the cancellous bone 63 and expose the diaphyseal cortical bone 62, a conical reamer with a pilot shaft matching the distal reamed diameter of the femoral stem 10 is used on the proximal end of the bone shaft to create a cavity that approximates the appropriate proximal stump size. When these systems and methods are used for placement of a hip implant, a broach is then used to expand the reamed cavity in the region of the medial calcar of the proximal femur, leaving the conical anteroposterior and lateral surfaces unaffected. A trial femoral stem may be attached to the broach to allow trial reduction of the reconstructed joint and selection of an appropriate size and shape for the head component. The broach is then removed and the femoral stem 10 is inserted. The femoral stem 10 can be preassembled with other components, for example, on a back table in the OR room or can be inserted first and then fitted with an appropriately dimensioned head 24 separately, without attaching them initially to the femoral stem 10.

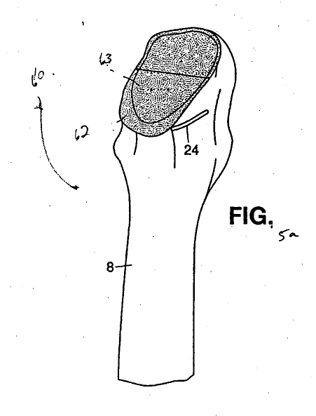

In a method of the present invention, the assembly, including the femoral stem 10 and rod 40, is implanted into the medullary canal 65 of the femur 60 during a first operation, as shown in FIG. 6. Since the rod 40 can have a smaller dimension than the exterior of the tapered portion 16, and still extend distally past the tapered portion 16, the required amount of cancellous bone 63 that must be removed is reduced without compromising the distal support of the femoral stem 10. The rod 40 is chosen such that when the rod is housed within the bore 28 and the cap 44 of the rod contacts the neck 20 of the femoral stem 10, the distal end 48 of the rod 40 contacts the bone surrounding the medullary canal, as shown in FIG. 6 at a desired location. Although the cap 44 is shown positioned above the femoral stem 10, the femoral stem may include a recess that receives the cap 44 so that the cap is flush with the proximal end of the tapered portion 16.

Unfortunately, once total hip arthroplasty has been performed, there may become a need to insert a device that extends distally further than either the femoral stem 10 or if the rod 40 extends past the femoral stem, to extend past the position of the distal end 48 of the rod. Situations where this may occur, include the requirement of further distal stabilization, treatment for a periprosthetic fracture and the like.

Prior to the present invention, if a periprosthetic fracture occurred the original implant has to be removed and replaced with a second implant. This included removing the entire femoral stem 10 from the femur and replacing the femoral stem with a second better suited implant. For example, as shown in FIG. 6, a periprosthetic fracture A has occurred in femur 60, distally past rod 40. The distal support for the femoral implant is now compromised which can lead to complications. Normally, this would require removal of the first femoral stem 10 from the femur 60 and implantation of a second femoral implant that extends deeper into the medullary canal, thus necessitating a second major surgery. Unfortunately, those who are most susceptible to this injury, the elderly, are those most in danger if a second surgery is required. In a method of the present invention, a second major surgery—along the lines of total hip replacement—can be eliminated.

Confronted with the same situation, as shown in FIGS. 6 and 7, the present femoral stem permits the removal of rod 40 from femoral stem 10, and replacement of the rod by a longer. i.e., further distally extending second rod.

Therefore, if a second operation is required, in a method of operation of the present invention, a surgeon makes an incision proximate the patient's hip. This incision is significantly smaller in size than that which would be required if replacement of the entire femoral stem was required. Rod 40, if present is than removed from femoral stem 10 via the incision. If required, various reaming, scraping and cleaning tools maybe inserted into the medullary canal of the femur via bore 28 of the femoral stem 10 so as to prepare the canal to receive a second rod.

As shown in FIG. 7, in a preferred embodiment, once the second rod 70 is placed within the bore 28 of the femoral stem 10, the second rod 70 extends distally past the periprosthetic fracture A. As is obvious, the second rod 70 also extends distally further than the distal end of femoral stem 10 as well as past the point where rod 40 had previously extended. The second rod 70 preferably contacts the bone of the femur surrounding the medullary canal. The location of the second rod 70 distally past the fracture A enables the second rod to distally support the femoral stem 10.

In an alternate embodiment, the second rod 70 may be inserted into the femoral stem using a retrograde approach. In such an instance, the distal end of the femur is exposed and resected to expose the canal. Various reamers and broaches are used to clean the medullary canal and specifically to remove much of the cancellous bone from the femur. With the femur now prepared, the second rod 40 may be received in the medullary canal at the distal end of the femur. The second rod 70 is translated towards the proximal end of the femur until the second rod 70 is positioned correctly within bore 28 of the femoral stem 10. Of course utilizing this method requires that the cap of the second rod not be larger than the bore 28 of the femoral stem in order that the second rod 70 may translate through the femoral stem 10.

The femoral stem of the present invention can be fabricated from any suitable high-strength biocompatible material. Such materials include titanium alloys, cobalt-chrome alloys, or stainless steel alloys and the like. Embodiments of the present invention may be adapted for use with or without surgical cement or other fixation elements.

The modular components of a prosthetic device according to the present invention are particularly well suited for inclusion in a kit that can be used by the surgeon to construct an implant specifically tailored to the patient's autonomy and dimensions. The kit may include a variety of components of different dimensions and shapes, including stem members, head members, and rod members from whom the surgeon can select a set of components dimensionally adapted for a particular patient. This allows the surgeon flexibility in assembling a complete prosthesis based on an individual patient's anatomy, either as determined at the time of surgery or determined in advance.

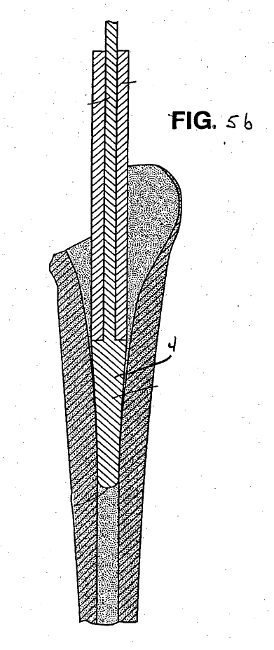

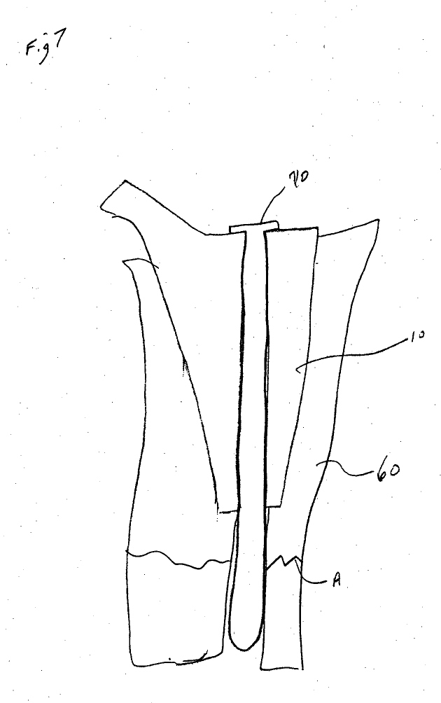

In alternate embodiments of the present invention, as shown in FIGS. 8-12, bore 28 may have a different configuration. With reference to FIG. 8, bore 128 of femoral stem 110 may include a tapered end 172. Although not shown in the figures, a corresponding tapered end may be included with rod 40 and/or second rod 70. The interaction between tapered end 172 of the bore 128 and tapered end of rods 40, 70 enable the rods to be mated to the femoral stem without being able to translate distally into the stem further than desired. Also, although not shown, bore 128 may be conically shaped and the rods 40, 70 designed with a corresponding structure.

As shown in FIG. 9, bore 228 may be non-symmetrical. A non-symmetrical bore may optimize stem stiffness against less stress-shielding induced bone resorption. The non-symmetrical bore also may provide better fitting options for retrograde entry and alignment. Retrograde alignment is the implantation of the rod or other prosthesis through the distal end of the femur as opposed to the proximal end.

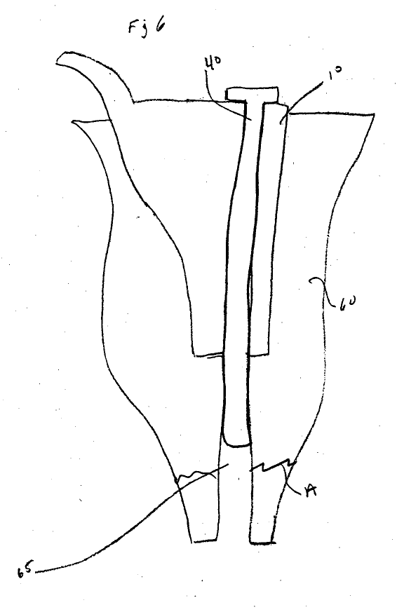

In yet another alternate embodiment, as shown in FIGS. 10a and 10b, a recess replaces the bore of the femoral stem. Recess 228 is positioned along an outside edge of the femoral stem 210 and is designed to receive a rod similar to previous embodiments discussed herein. Thus, when rod 40, 70 is positioned relative to femoral stem 210 in a femur, the rod is not completely surrounded by an interior wall of the femoral stem but rather is positioned between the femoral stem and the femur. This enables the rods to be positioned off-set as compared to the medullary canal if so required.

In yet another alternate embodiment of the present invention, the femoral stem may have more than one bore. For instance, as shown in FIG. 11, femoral stem 310 includes a first bore 328 and a second bore 329. Bores 328, 329 may be parallel or slightly off-set from one another. Each bore 328, 329 is adapted to receive a first rod which is replaceable with a second rod as discussed in conjunction with prior embodiments. The use of two rods increases distal support of the femoral stem.

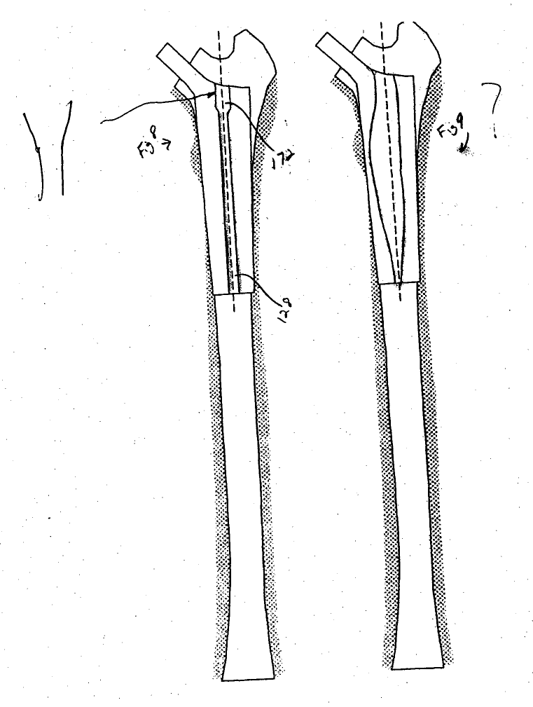

In still another alternate embodiment of the present invention, rod 440, as shown in FIG. 13 may include at least two tines 441, 442 disposed at the distal end 415 of the rod. The tines 441, 442 provide greater support and an additional anchoring mechanism for the femoral stem 410 with the femur. Various mechanisms known to those in the art may be provided in order to enable tines 441, 442 to expand outwardly after the rod 40, 70 has been positioned within the femoral stem 10. The tines 441, 442 are expanded outwardly so as to engage the interior bone of the femur and provide greater stability.

As shown in FIG. 14, femoral stem 510 and rod 540 may be adapted for use in conjunction with fixation elements such as cross locking screws 580. Fixation elements 580 may include nails, screws or the like. The fixation elements 580 are designed to secure the femur 60 to the femoral stem 510. For instance, a situation may arise wherein the femur 60 is fractured at point B. By securing the femur 60 to the femoral stem 510, the femur remains relatively stable at both points above and below the fracture B. With both points of the femur above and below the fracture immobilized, the risk of complications arising during the healing of the femur is reduced.

In order to best affix the femur 60 to the rod 540 and/or femoral stem 510, the rod may be provided with at least one aperture 572 extending from a first side 573 of the rod to a second side 574. The fixation elements 580 are driven through the femur 60 in a method known by those in the art and received within the apertures 572 of the rod 540. Targeting instruments similar to those used with 1M nails may be utilized.

Of course, if there should come a need to replace rod 540 with a second rod, as earlier discussed, fixation elements 580 are simply removed from engagement with the femoral stem 510. The rod 540 is subsequently removed and replaced with a second rod 570. The fixation elements 580 may also be refitted with the second rod 570 in place.

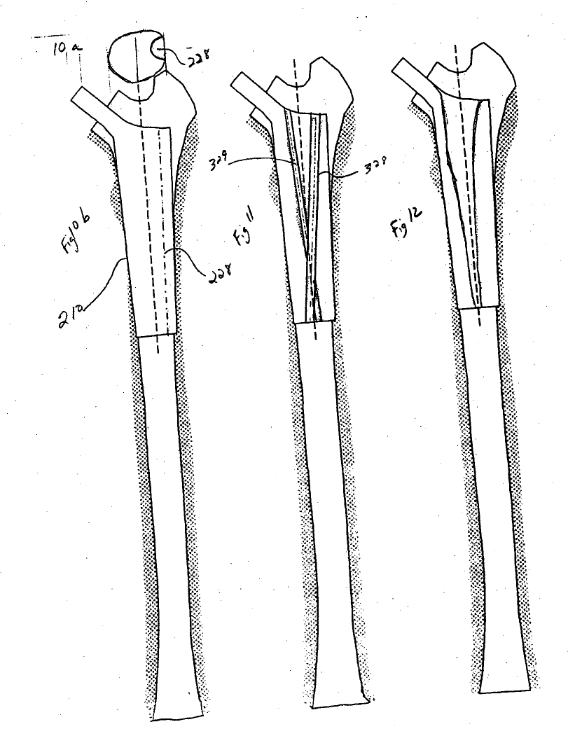

In yet another aspect of the present invention, an alignment may be used to position the rod correctly with the femoral stem. For instance, as shown in FIG. 15, the alignment guide 690 may include a rounded surface 645 disposed at the bottom of cap 644. Rounded surface 645 may include roughened surface 646.

In a method of use the proximal end of the femoral stem 610 is shaped so as to include a hemispherical recess 647 for mating with the hemispherical recess i.e. rounded surface 645, of rod 640. The rod 640 may be pivoted along the hemispherical recess 647 of the femoral stem until the shaft 642 is correctly aligned. Once the shaft 642 is correctly aligned, the cap 644 may be compressed into the hemispherical recess 647 so that the roughened surface 645 of the cap 644 engages the femoral stem surface defining the hemispherical recess 647. The hemispherical surface 647 of the femoral stem may also include a roughened surface to interact with the roughened surface of the rounded surface 645 of rod 640.

In an alternate method, as shown in FIG. 16, an alignment guide 790 may include an arcuate surface 791 having a flange 792 extending outwardly from remote ends of the arcuate surface. Flanges 792 may be secured to the proximal end of femoral stem 710 with securing elements 793 so that arcuate surface 792 remains affixed relative to the femoral stem. Rod 740 is attached to the arcuate surface 792 through an adjusting device 795. The adjusting device 795 permits the pivoting of the rod 740 in an X-direction and a Y-direction in order that the rod may be correctly aligned. Once the shaft 742 of rod 740 is correctly aligned with the medullary canal of femur 60, the rod may be disconnected from the arcuate surface 792 and remain correctly positioned within the bore 728 of the femoral stem 710. At this point, securing elements 793 may be disassembled from the femoral stem 710 and the entire alignment guide 790 removed.

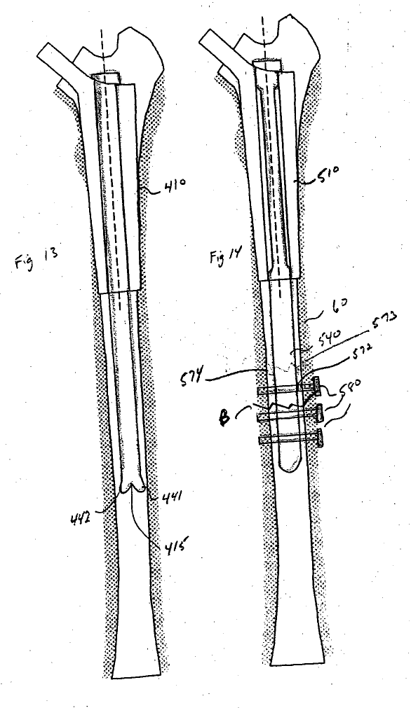

In yet another alternate embodiment of the present invention, as shown in FIG. 17, alignment guide 890 may include a shelf 892 that is connectable to the femoral stem 810 by the use of securing elements 893. An adjusting device 894 such as a rod, screw or the like is positioned within a bore 895 of the shelf 892. The adjusting device is able to engage rod 840 such that the two are connected. The adjusting device 894 may include an adjusting handle 896 so as to simplify the moving of the adjusting device 894 for a user. The adjusting handle 896 may be connected to the adjusting device 894 using mating internal and external threads or the like. The bore 895 is slightly larger than the adjusting device 894 so that the adjusting device can move freely within the bore. However, a flange 897 extends about the bore 895 in order that the adjusting device 894 may not fall through the shelf 892.

As the adjusting handle 896 is moved back and forth, the adjusting device 894 rotates, rocks and translates within bore 895. This movement subsequently results in movement of the rod 840. In this manner, the rod 844 may be correctly aligned with the femoral stem 810 and femur. Once the rod 840 is correctly placed, the alignment guide 890 may be removed by disassembling the securing elements 893 for the femoral stem 810 and disassembly the adjusting device 894 from the rod 840.

In yet another alternate embodiment of the present invention as shown in FIG. 18, alignment guide 990 may include two flanges 991 attached to femoral stem 910 and a base 992 extending between the flanges. A pivot rod 993 extends outwardly from base 992 and is positioned above bore 928. Rod 940 is attached to pivot rod 993 and extends downwardly therefrom into bore 928. The rod 940 may be capable of rotating about pivoting rod 993 and/or translate back and forth, i.e. into and out of the page relative to the reader, about the pivoting rod.

In this manner, the rod 940 may be correctly positioned within the bore 928 of the femoral stem 910. Once correctly positioned the rod 940 may be locked into place.

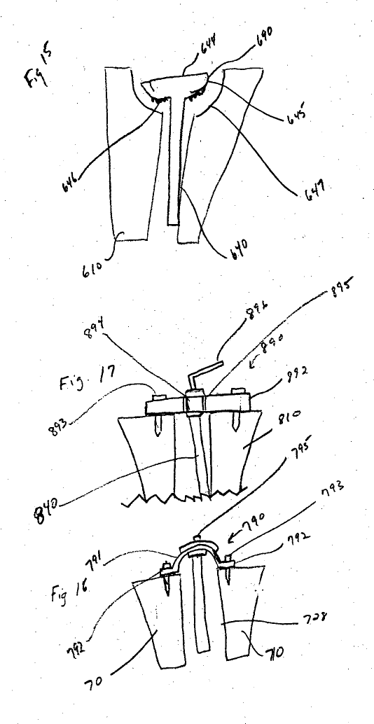

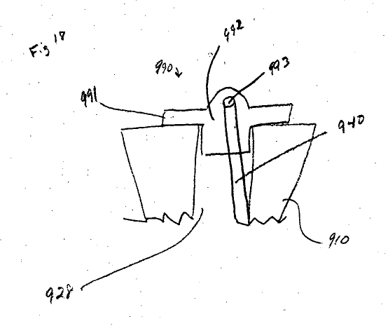

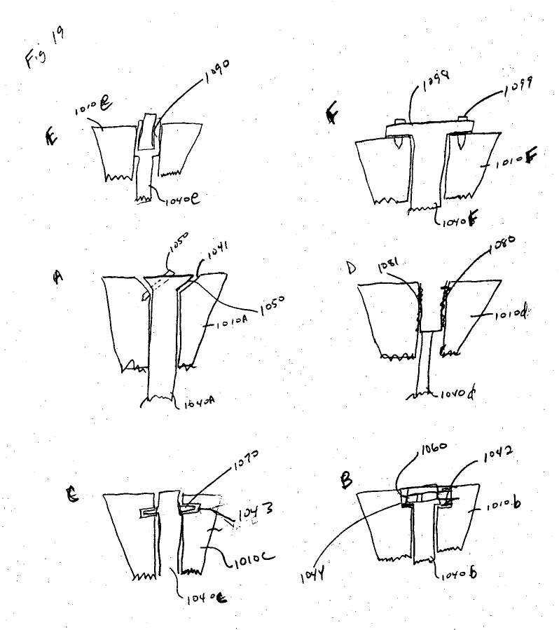

As shown in FIGS. 19a-f, the rods of the present invention may be fitted with a coupling mechanism for coupling the rod 1040 to the stem 1010. The stems 1010 may have tapered surfaces 1041, shelves 1042 or indents 1043 for allowing the anchoring of the rod 1040 to the stem.

In the embodiment as shown in FIG. 19a, the rod 1040a has a matching tapered surface 1050 which is positioned adjacent to the tapered surface 1041 of the stem 1010a. A screw or nail 1050 may also be used to securely attach the two elements.

In the shelf embodiment, as shown in FIG. 19b, the rod 1040b may have a locking ring 1060 which locks into recess 1044 of stem 1010b. Locking rings of this sort are known to those in the art.

In the indent embodiment, as shown in FIG. 19c, the stem 1010c includes an indent 1043 about a bore of the stem. The rod 1040c has a spring clip or similar device 1070 that enables the rod 1040c to be slidably received within the bore of the stem 1010e. When the spring clip 1070 is positioned adjacent to the indent 1043, the spring clip expands and is positioned within the indent thereby locking the rod 1040c to the stem 1010c. In a preferred embodiment, the spring clip can be closed allowing the rod to be slidably removed from the stem 1010c.

In still alternate embodiments, the coupling piece may take the form of threaded mating surfaces 1080 and 1080 such as that shown in FIG. 19d or the rod may have an expandable proximal end 1090 as shown in FIG. 19e. Once the rod 1040e has been slidably received within the stem 1010e a wedge 1090 is placed within a recess 1092 of the rod. The wedge may causes the end of the rod 1040e to expand outward until the rod 1040e is locked within the stem 1010e.

In an alternate embodiment the rod 1040f may have a cap that is either screwed or nailed to the stem 1010f with fixation devices 1099.

Of course any combination of the above mentioned embodiments is contemplated by the present invention.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A joint prosthesis kit comprising:
a plurality of stems, each having a first side, a second side, a proximal end, a distal end and a bore extending from said proximal end to said distal end, said bore being defined by an interior wall of said stem, said stem being insertable within a medullary canal of a bone;
a first rod having a distal tip capable of being inserted into the bore from the proximal end of said stems and housed in said bore, said distal tip of said first rod extending past said distal end of said stems such that said first rod extends farther into the medullary canal of the bone than said stems;
a second rod having a distal tip capable of being inserted into the bore from the proximal end of said stem, said first rod being replaceable by said second rod, said distal tip of said second rod extending distally farther into the medullary canal of the bone when said second rod is disposed within said stem and the medullary canal as compared to said distal tip of said first rod when said first rod is disposed within said stem and the medullary canal; and
a coupling element for coupling said first and second rods to said proximal end of said stem.

2. The joint prosthesis kits according to claim 1, wherein said stem is implanted into the medullary canal of a femur.

3. The joint prosthesis kits according to claim 1, further comprising a neck and a head, said neck being disposed at said proximal end of said stem, said head extending outwardly from said neck and away from said distal end of said stem.

4. The joint prosthesis kit according to claim 3, wherein said head is recoverably attached to said neck.

5. The joint prosthesis kit according to claim 1, wherein said first rod includes a proximal end and a cap positioned near said proximal end of said cap, wherein when said first rod is placed within said bore of said stem said cap abuts said proximal end of said stem to limit a depth to which said first rod may extend into the medullary canal of the bone.

6. The joint prosthesis kit according to claim 1, wherein said interior wall of said stem includes a cylindrical portion.

7. The joint prosthesis according to claim 1, wherein said bore of said stem includes a tapered portion.

8. The joint prosthesis kit according to claim 1, wherein said bore of said stem includes a non-symmetrical portion.

9. The joint prosthesis kit according to claim 1, wherein said at least one stem includes a second interior wall defining a second bore, said second bore being capable of receiving a third rod disposed therein.

10. The joint prosthesis kit according to claim 9, wherein said third rod is constructed so as to be replaceable by a fourth rod after said stem is implanted into a human body.

11. The joint prosthesis kit according to claim 1, wherein said first rod has a first longitudinal axis and said bore has a second longitudinal axis, wherein said first longitudinal axis and said second longitudinal axis are substantially perpendicular to one another when said first rod is disposed within said bore.

12. The joint prosthesis kit according to claim 1, further comprising at least on fixation element, wherein said first rod includes at least one aperture extending from said first side to said second side, wherein said at least one fixation element is adapted to secure the bone to said stem by said at least one fixation element extending through the bone and into said at least one aperture of said stem.

13. A method of operating on a joint having a medullary canal, comprising the steps of:
removing a first rod disposed within a bore of a joint prosthesis, said first rod including a distal tip that extends past said joint prosthesis when said joint prosthesis is implanted in the medullary canal of a bone; and
placing a second rod in said bore of said joint prosthesis, said second rod having a second distal tip that extends distally further into the medullary canal of the bone as compared to said distal tip of said first rod when said first rod is positioned within said joint prosthesis and the medullary canal of the bone.

14. The method according to claim 13, wherein the step of removing said first rod includes slidably removing said first rod from said joint prosthesis.

15. The method according to claim 13, wherein said joint prosthesis is a hip implant.

16. The method according to claim 13, wherein said step of placing said second rod in said bore of said joint prosthesis includes introducing said second rod into said bore of said joint prosthesis at the proximal end of said joint prosthesis and translating said second rod from said proximal end of said stem in a direction towards said distal end of said joint prosthesis.

17. The method according to claim 13, wherein said bore of said joint prosthesis is tapered and said second rod has a corresponding tapered shape for communicating with said bore.

18. The method according to claim 13, further comprising providing at least one fixation element and affixing said first rod to the bone by pinning the bone to said first rod with said at least one fixation element.

19. The method according to claim 13, further comprising providing at least one fixation element and affixing said second rod to the bone by pinning the bone to said second rod with said at least one fixation element.

20. The method according to claim 13, further comprising reaming the medullary canal prior to placement of said second rod.
